(19) United States
(12) Patent Application Publication
(10) Pub. No.: US 2015/0125915 A1
(43) Pub. Date: May 7, 2015

(54) Method for Producing Lactic Acid

(71) Applicant: Kao Corporation, Chuo-ku, Tokyo (JP)

(72) Inventors: Yuichi Tsuboi, Wakayama-shi (JP); Fumikazu Takahashi, Wakayama-shi (JP); Yumi Nishimura, Wakayama-shi (JP); Kazuhisa Sawada, Haga-gun (JP)

(73) Assignee: Kao Corporation, Chuo-ku, Tokyo (JP)

(21) Appl. No.: 14/394,378

(22) PCT Filed: Apr. 4, 2013
(86) PCT No.: PCT/JP2013/060305

§ 371 Date: Oct. 14, 2014

(30) Foreign Application Priority Data
Apr. 19, 2012 (JP) 2012-095709

(51) Int. Cl. C12P 7/56; C12N 1/14

(52) U.S. Cl. CPC C12P 7/56; C12N 1/14
USPC 435/139; 435/256.6

(57) ABSTRACT

It is intended to provide a fungus of the genus Rhizopus having improved ability to produce lactic acid and a method for producing lactic acid using the fungus. The present invention provides a method for improving the lactate dehydrogenase activity of a fungus of the genus Rhizopus, comprising germinating a spore of a fungus of the genus Rhizopus in a culture medium containing a surfactant under specific conditions to obtain a mycelium, and a method for producing lactic acid using the mycelium of the fungus of the genus Rhizopus.

### FIELD OF THE INVENTION

The present invention relates to a method for producing lactic acid.

### BACKGROUND OF THE INVENTION

Lactic acid, which serves as a starting material for poly-lactic acid (PLA) receiving attention as a biodegradable plastic material, is obtained by a biological production method from a plant-derived starting material. For this reason, PLA is regarded as a material which does not increase CO₂ levels in the air (carbon-neutral material). The lactic acid includes optical isomers D-form and L-form. Accordingly, highly optically pure lactic acid is required for controlling the properties of poly-lactic acid as a plastic material.

A conventionally known method for biologically producing lactic acid involves anaerobically culturing a lactic acid bacterium belonging to the genus Lactobacillus, the genus Lactococcus, or the like to cause lactic acid fermentation (Non Patent Literature 1). In addition, a method which involves culturing a filamentous fungus of the genus Rhizopus is known as a method for specifically producing L-lactic acid (Patent Literatures 1 to 3). The lactic acid production using the fungus of the genus Rhizopus is less than satisfactory in terms of production rate, in spite of its advantages that, for example, highly optically pure lactic acid is obtained and culture is achieved using relatively simple medium composition.

Filamentous fungi are generally cultured by: forming spores through sufficient growth in a solid medium such as a slant in order to control an inoculum dose; dispersing the spores in sterile water or the like using a small amount of a surfactant; and inoculating a fixed number of spores per unit medium (e.g., Patent Literatures 2 and 4). Solid culture or liquid culture is known as a general method for culturing filamentous fungi. Liquid culture is predominantly used in matter production using the genus Rhizopus. In this respect, various forms including filamentous, massive, and pellet forms are used as fungal forms (Non Patent Literature 2 and Patent Literatures 2 to 5).

Medium conditions, culture conditions, and the like for the lactic acid production using the fungus of the genus Rhizopus have been studied in various ways in order to improve its ability to produce lactic acid (Non Patent Literature 3 and Patent Literatures 3, 4, and 6). Non Patent Literature 4 discloses the correlation of the ability to produce lactic acid with lactate dehydrogenase (LDH) activity and a successful improvement of the ability to produce lactic acid by means of the high expression of LDH introduced in Rhizopus oryzae by a gene recombination technique. However, a method for improving the LDH activity of a fungus of the genus Rhizopus in a manner independent of gene recombination has not yet been revealed.

### CITATION LIST

### Patent Literature

[Patent Literature 1] JPS51-12990 A
[Patent Literature 2] JP2000-037196 A
[Patent Literature 3] JP2006-246846 A
[Patent Literature 4] JPH06-253871 A
[Patent Literature 5] JPH09-077803 A
[Patent Literature 6] U.S. Pat. No. 4,564,594
[Non Patent Literature 1] Enzyme and Microbial Technology (2000) vol. 26: 87-107
[Non Patent Literature 2] Seibutsu-kogaku Kaishi (2000) vol. 78, No. 12: 487-493
[Non Patent Literature 3] Applied Biochemistry and Biotechnology (1999) vol. 78: 401-407
[Non Patent Literature 4] Applied Microbiology and Biotechnology (2004) vol. 64: 237-242

### SUMMARY OF THE INVENTION

Specifically, the present invention provides a method for improving the lactate dehydrogenase (LDH) activity of a fungus of the genus Rhizopus, comprising the following step (A1):
(A1) germinating a spore of a fungus of the genus Rhizopus in a first culture medium comprising 0.01% (w/v) or higher of a surfactant to obtain a mycelium, wherein the surfactant is at least one surfactant selected from the group consisting of sorbitan fatty acid ester, POE sorbitan fatty acid ester, POE alkyl ether having an EO addition mole number of 5 or less, deoxycholate, alkenyl succinate, and polyoxyethylene alkyl phenyl ether.

The present invention also provides a method for producing a fungus of the genus Rhizopus having improved ability to produce lactic acid, comprising obtaining a fungus of the genus Rhizopus having improved lactate dehydrogenase activity by the method for improving the LDH activity according to the present invention.

The present invention further provides a fungus of the genus Rhizopus obtained by the production method.

The present invention further provides a method for producing lactic acid, comprising the following steps (A1) and (B):
(A1) germinating a spore of a fungus of the genus Rhizopus in a first culture medium comprising 0.01% (w/v) or higher of a surfactant to obtain a mycelium having improved lactate dehydrogenase activity; and
(B) further culturing the obtained mycelium in a third culture medium to obtain lactic acid, wherein the surfactant is at least one surfactant selected from the group consisting of sorbitan fatty acid ester, POE sorbitan fatty acid ester, POE alkyl ether having an EO addition mole number of 5 or less, deoxycholate, alkenyl succinate, and polyoxyethylene alkyl phenyl ether.

### DETAILED DESCRIPTION OF THE INVENTION

The present invention relates to a method for improving the lactate dehydrogenase (LDH) activity of a fungus of the genus Rhizopus or the ability thereof to produce lactic acid, a fungus of the genus Rhizopus having improved LDH activity or ability to produce lactic acid which is obtained by the method, and a method for efficiently producing lactic acid using the fungus of the genus Rhizopus.

The present inventors have studied a method for improving the ability of a fungus of the genus Rhizopus to produce lactic acid in a manner independent of an artificial gene recombination technique. As a result, the present inventors found that a fungus of the genus Rhizopus having a mycelium germinated and grown from a spore in a medium containing a surfactant under specific conditions exhibits high LDH activity and further has the high ability to produce lactic acid. The present inventors also found that the fungus of the genus Rhizopus can be cultured to thereby efficiently produce lactic acid.

The fungus body of the genus Rhizopus obtained by the present invention has high LDH activity and also has the improved ability to produce lactic acid. The fungus body can be cultured to thereby efficiently produce lactic acid.

According to one aspect, the present invention provides a method for improving the LDH activity of a fungus of the genus Rhizopus. LDH is an enzyme which catalyzes the conversion of pyruvate to lactate. It has been reported that a fungus of the genus Rhizopus having high LDH activity produces a larger amount of lactic acid. The fungus of the genus Rhizopus obtained by the present invention has high LDH activity and the high ability to produce lactic acid and is useful as a fungus of the genus Rhizopus for lactic acid production.

Examples of the fungus of the genus Rhizopus which is subjected to the method for improving the LDH activity according to the present invention include fungi of the genus Rhizopus naturally having LDH activity and fungi of the genus Rhizopus naturally having the ability to produce lactic acid. From the viewpoint of improving the LDH activity or improving the ability to produce lactic acid, preferred examples of the fungus of the genus Rhizopus which is subjected to the method of the present invention include Rhizopus oryzae, Rhizopus arrhizus, Rhizopus chinensis, Rhizopus nigricans, Rhizopus tonkinensis, and Rhizopus tritici. Alternatively, the fungus of the genus Rhizopus which is subjected to the method for improving the LDH activity according to the present invention may be a fungus of the genus Rhizopus naturally having no or a little LDH activity or ability to produce lactic acid, but artificially modified to improve the LDH activity or the ability to produce lactic acid. Examples of such a fungus of the genus Rhizopus include fungus strains of the genus Rhizopus which were constructed by transferring the IdhA gene into the genus Rhizopus having the low ability to produce lactic acid. Of the fungi of the genus Rhizopus listed above, Rhizopus oryzae is more preferred from the viewpoints of improvement in LDH activity and the ability to produce lactic acid and easy availability. Examples of preferred fungus strains of Rhizopus oryzae include Rhizopus oryzae NBRC 4707, NBRC 4785, NBRC 5384, and NBRC 5418 deposited and registered in Biological Resource Center (NBRC), National Institute of Technology and Evaluation (NITE), which is a culture collection. These fungus strains are available from NERC as well as Riken BioResource Center (BRC), etc.

The method for improving the LDH activity according to the present invention comprises the step of germinating a spore of any above-mentioned fungus of the genus Rhizopus desired to improve LDH activity or lactic acid productivity in a first culture medium comprising 0.01% (w/v) or higher of a surfactant to obtain a mycelium (hereinafter, also referred to as “step (A1)”).

The fungus of the genus Rhizopus which is subjected to the step (A1) is preferably wholly in the form of spores and may contain hyphae. The spores of the fungus of the genus Rhizopus can be prepared, for example, by: inoculating a medium with a suspension containing spores of the above-mentioned fungus of the genus Rhizopus, followed by static culture; visually confirming hyphal growth and sporulation; then suspending the cultures in a liquid containing a surfactant; and after standing, recovering the supernatant as a spore suspension. The number of spores in the recovered spore suspension can be counted using a hemocytometer or the like under microscopic observation. The spore suspension can be adjusted to the desired number of spores by appropriate dilution.

The step (A1) involves inoculating the spore suspension thus obtained to the first culture medium, and germinating the spores by culture to obtain mycelia. The number of spores of the fungus of the genus Rhizopus to be inoculated to the culture medium is preferably from 1×10² to 5×10⁴ spores/mL of the culture medium, more preferably from 5×10² to 1×10⁴ spores/mL of the culture medium, further preferably from 1×10³ to 1×10⁴ spores/mL of the culture medium. A commercially available medium, for example, a potato dextrose medium (hereinafter, referred to as a PDB medium; manufactured by, for example, Becton, Dickinson and Company), a Luria-Bertani medium (hereinafter, referred to as an LB medium; for example, “Daigo” manufactured by Nihon Pharmaceutical Co., Ltd.), a nutrient broth (hereinafter, referred to as an NB medium; manufactured by, for example, Becton, Dickinson and Company), or a Sabouraud medium (hereinafter, referred to as an SB medium; manufactured by, for example, Oxoid Ltd.) can be used as the first culture medium for spore germination in the step (A1). If necessary, the first culture medium used in the step (A1) can be appropriately supplemented with a carbon source such as monosaccharides including glucose and xylose, oligosaccharides including sucrose, lactose, and maltose, polysaccharides including starch or biogenic substances including glycerin and citric acid, a nitrogen source such as ammonium sulfate, urea and an amino acid or the like, and other inorganic materials including various salts of sodium, potassium, magnesium, zinc, iron, phosphoric acid, or the like, from the viewpoints of the rate of germination and fungus body growth. The concentration of a monosaccharide, an oligosaccharide, a polysaccharide, or glycerin is preferably from 0.1 to 30% (w/v). The concentration of citric acid is preferably from 0.01 to 10% (w/v). The concentration of ammonium sulfate, urea, or amino acid is preferably from 0.01 to 1% (w/v). The concentration of an inorganic material is preferably from 0.0001 to 0.5% (w/v).

The first culture medium used in the step (A1) contains at least one surfactant selected from the group consisting of sorbitan fatty acid ester, polyoxyethylene (POE) sorbitan fatty acid ester, polyoxyethylene (POE) alkyl ether having an ethylene oxide (EO) average addition mole number of 5 or less, deoxycholate, alkenyl succinate, and polyoxyethylene alkyl phenyl ether. The spores of the fungus of the genus Rhizopus are cultured into mycelia in the presence of the surfactant to thereby improve the LDH activity of the fungus and the ability thereof to produce lactic acid. From the viewpoints of improving the LDH activity and the ability to produce lactic acid and preventing foaming during culture, the surfactant is preferably at least one surfactant selected from the group consisting of sorbitan monolaurate, sorbitan monooleate, polyoxyethylene sorbitan monolaurate having an EO average addition mole number of 20 or less, polyoxyethylene lauryl ether having an EO average addition mole number of 5 or less, deoxycholate, alkenyl succinate, and polyoxyethylene octyl phenyl ether having an EO average addition mole number of 10, more preferably at least one surfactant selected from the group consisting of sorbitan monolaurate, sorbitan monooleate, polyoxyethylene sorbitan monolaurate having an EO average addition mole number of 4, polyoxyethylene lauryl ether having an EO average addition mole number of 3 or 4, and alkenyl succinate, further preferably sorbitan monolaurate.

A commercially available product can be purchased as the surfactant. Specific examples thereof can include: Rheodol(R) SP-L10 and Rheodol(R) Super SP-L10 (all manufactured by Kao Corp.) as sorbitan monolaurate; Rheodol(R) SP-010V as sorbitan monooleate; Rheodol(R) TW-L106 and Rheodol(R) Super TW-L120 (all manufactured by Kao Corp.) as polyoxyethylene sorbitan monolaurate having an EO average addition mole number of 20 or less; Emulgen(R) 103, Emulgen(R) 104P, and Emulgen(R) 106 (all manufactured by Kao Corp.) as polyoxyethylene lauryl ether having an EO average addition mole number of 5 or less; sodium deoxycholate (manufactured by Wako Pure Chemical Industries Ltd.) as deoxycholate; Latemul(R) ASK (manufactured by Kao Corp.) as alkenyl succinate; and TRITON(R) X-100 (MP Biomedicals, LLC) as polyoxyethylene alkyl phenyl ether.

The first culture medium used in the step (A1) can be supplemented with at least one surfactant selected from the group consisting of the surfactants mentioned above. From the viewpoint of improving the LDH activity and the ability to produce lactic acid, the concentration of the surfactant in the first culture medium is 0.01% (w/v) or higher, preferably 0.05% (w/v) or higher, more preferably 0.1% (w/v) or higher, further preferably 0.2% (w/v) or higher, in terms of the final concentration in the culture medium. From a similar viewpoint, the final concentration of the surfactant in the culture medium is preferably lower than 2.5% (w/v), more preferably lower than 1.5% (w/v), further preferably lower than 1.0% (w/v), still further preferably lower than 0.8% (w/v). From the viewpoint of improving the LDH activity and the ability to produce lactic acid, the concentration of the surfactant in the culture medium used in the step (A1) is preferably 0.01% (w/v) or higher and lower than 2.5% (w/v), more preferably 0.05% (w/v) or higher and lower than 1.5% (w/v), further preferably 0.1% (w/v) or higher and lower than 1.0% (w/v), still further preferably 0.2% (w/v) or higher and lower than 0.8% (w/v), in terms of the final concentration in the culture medium.

In the step (A1), the fungus of the genus Rhizopus is cultured using the first culture medium containing the surfactant. This culture can be performed by usual procedures. For example, the spores of the fungus of the genus Rhizopus are inoculated to a culture vessel containing the first culture medium containing the surfactant and then cultured at a culture temperature controlled from 25 to 42.5° C. for preferably from 24 to 120 hours, more preferably from 48 to 72 hours, with stirring at preferably from 80 to 250 rpm, more preferably from 100 to 170 rpm. The amount of the first culture medium subjected to the culture can be appropriately adjusted according to the size of a culture vessel and can be, for example, on the order of from 50 to 100 mL for a 200-mL baffled flask and on the order of from 100 to 300 mL for a 500-mL baffled flask. By this culture, the spores of the fungus of the genus Rhizopus are germinated and grown into mycelia.

From the viewpoint of improving the LDH activity and the ability to produce lactic acid, the method for improving the LDH activity according to the present invention preferably further comprises the step of growing the mycelium obtained in the step (A1) by further culture (hereinafter, also referred to as step (A2)). The culture medium for growth used in the step (A2) (hereinafter, also referred to as a “second culture medium”) is not particularly limited and can be a glucose-containing inorganic culture medium usually used. Examples thereof include a culture medium containing from 7.5 to 30% glucose, from 0.05 to 2% ammonium sulfate, from 0.03 to 0.6% potassium dihydrogen phosphate, from 0.01 to 0.1% magnesium sulfate heptahydrate, from 0.005 to 0.05% zinc sulfate heptahydrate, and from 3.75 to 20% calcium carbonate (all the concentrations mean % (w/v)) and preferably include a culture medium containing 10% glucose, 0.1% ammonium sulfate, 0.06% potassium dihydrogen phosphate, 0.025% magnesium sulfate heptahydrate, 0.009% zinc sulfate heptahydrate, and 5.0% calcium carbonate (all the concentrations mean % (w/v)). The amount of the second culture medium can be appropriately adjusted according to the size of a culture vessel and can be, for example, from 50 to 300 mL, preferably from 100 to 200 mL, for a 500-mL baffled flask. The mycelia of the fungus of the genus Rhizopus obtained by the culture in the step (A1) are inoculated to this culture medium at a dose of from 1 to 6 g of the fungus bodies/100 mL of the medium, preferably from 3 to 4 g of the fungus bodies/100 mL of the medium, in terms of wet weight and then cultured at a culture temperature controlled to from 25 to 42.5° C. for from 12 to 120 hours, preferably from 24 to 72 hours, with stirring at from 100 to 300 rpm, preferably from 170 to 230 rpm.

The second culture medium used in the step (A2) does not have to contain the surfactant mentioned above in relation to the first culture medium, but may contain the surfactant. From the viewpoint of improving the LDH activity and the ability to produce lactic acid, the concentration of the surfactant in the second culture medium is preferably ⅕ or lower, more preferably 1/10 or lower, further preferably 1/20 or lower, of the concentration of the surfactant in the first culture medium used in the step (A1). From the viewpoint of improving the LDH activity and the ability to produce lactic acid, the concentration of the surfactant in the second culture medium used in the step (A2) is, for example, preferably 0.2% (w/v) or lower, more preferably 0.15% (w/v) or lower, further preferably 0.1% (w/v) or lower, still further preferably 0.05% (w/v) or lower, particularly preferably 0.01% (w/v) or lower, in terms of the final concentration in the culture medium.

The mycelia of the fungus of the genus Rhizopus obtained by the above step (A1) or step (A2) preferably have a pellet form. Pellet composed of individual particles which are clearly independent and have smooth surface, a particle size on the order of from 0.5 to 5 mm, and substantially uniform shapes is more preferred because such pellet is easy to handle during the process of lactic acid production mentioned later and has a shape hard to destroy even by repetitive use. Pellet having the desired size or appearance can be formed by changing the culture time, the culture temperature, the stirring rate during culture, or the like. The method of the present invention, however, does not necessarily require pelletizing the mycelia or does not necessarily require adjusting the size or appearance of the pellet.

Subsequently, each mycelium obtained by the above step (A1) or step (A2) is recovered as a fungus of the genus Rhizopus having improved LDH activity. The method for recovering the mycelium is not particularly limited, and the mycelium can be recovered by a usual method such as decantation, filtration, or centrifugation.

The fungus of the genus Rhizopus thus obtained by the method for improving the LDH activity of a fungus of the genus Rhizopus according to the present invention can have the higher ability to produce lactic acid by virtue of its improved LDH activity. Thus, according to another aspect, the present invention provides a method for improving the ability of a fungus of the genus Rhizopus to produce lactic acid, comprising the above step (A1) and preferably further comprising the above step (A2).

The method for improving the LDH activity of a fungus of the genus Rhizopus according to the present invention can yield a fungus body of the genus Rhizopus having improved LDH activity and ability to produce lactic acid. This fungus of the genus Rhizopus is useful in efficient lactic acid production. Thus, according to a further alternative aspect, the present invention provides a method for producing a fungus of the genus Rhizopus having improved LDH activity or ability to produce lactic acid, comprising performing the above step (A1) and preferably further performing the above step (A2) to obtain a mycelium of the fungus of the genus Rhizopus having improved LDH activity or ability to produce lactic acid. According to a further alternative aspect, the present invention provides a fungus of the genus Rhizopus having improved LDH activity or ability to produce lactic acid which is obtained by performing the above step (A1) and preferably further performing the above step (A2).

According to a further alternative aspect, the present invention provides a method for producing lactic acid, further comprising the step of culturing the mycelium of the fungus of the genus Rhizopus obtained by the above step (A1) and preferably further the step (A2) (hereinafter, also referred to as “step (B)”). In the method for producing lactic acid according to the present invention, lactic acid can be obtained by culturing the mycelium of the fungus of the genus Rhizopus having improved LDH activity or ability to produced lactic acid, obtained by the above procedures, and then by recovering the lactic acid produced from the culture.

The culture medium for lactic acid production used in the step (B) (hereinafter, also referred to as a “third culture medium”) can be any culture medium which contains a carbon source such as glucose, a nitrogen source such as ammonium sulfate, and various metal salts, etc. and permits production of lactic acid. Examples of the third culture medium used in the step (B) include a culture medium containing from 7.5 to 30% glucose, from 0.05 to 2% ammonium sulfate, from 0.03 to 0.6% potassium dihydrogen phosphate, from 0.01 to 0.1% magnesium sulfate heptahydrate, from 0.005 to 0.05% zinc sulfate heptahydrate, and from 3.75 to 20% calcium carbonate (all the concentrations mean % (w/v)) and preferably include a culture medium containing 12.5% glucose, 0.1% ammonium sulfate, 0.06% potassium dihydrogen phosphate, 0.025% magnesium sulfate heptahydrate, 0.009% zinc sulfate heptahydrate, and 5.0% calcium carbonate (all the concentrations mean % (w/v)).

The amount of the third culture medium used in the step (B) can be appropriately adjusted according to the size of a culture vessel and can be, for example, on the order of from 20 to 80 mL for a 200-mL Erlenmeyer flask and on the order of from 50 to 200 mL for a 500-mL Erlenmeyer flask. The above-mentioned fungus of the genus Rhizopus having improved LDH activity or ability to produce lactic acid is inoculated to this third culture medium at a dose of from 10 g to 90 g of the fungus bodies/100 mL of the culture medium, preferably from 15 g to 50 g of the fungus bodies/100 mL of the culture medium, in terms of wet weight and then cultured at a culture temperature controlled to from 25 to 45° C. for from 4 hours to 24 hours, preferably from 6 hours to 12 hours, with stirring at from 100 to 300 rpm, preferably from 170 to 230 rpm.

The third culture medium used in the step (B) does not have to contain the surfactant mentioned above in relation to the first culture medium, but may contain the surfactant. From the viewpoint of improving the LDH activity and the ability to produce lactic acid, the concentration of the surfactant in the third culture medium is preferably ⅕ or lower, more preferably 1/10 or lower, further preferably 1/20 or lower, of the concentration of the surfactant in the first culture medium used in the step (A1). From the viewpoint of improving the LDH activity and the ability to produce lactic acid, the concentration of the surfactant in the culture medium used in the step (B) is, for example, preferably 0.2% (w/v) or lower, more preferably 0.15% (w/v) or lower, further preferably 0.1% (w/v) or lower, still further preferably 0.05% (w/v) or lower, particularly preferably 0.01% (w/v) or lower, in terms of the final concentration in the culture medium.

The culture supernatant can be recovered from the third culture medium to obtain lactic acid. If necessary, the lactic acid in the culture medium is recovered as lactate by a method such as decantation, membrane separation, centrifugation, electrodialysis, utilization of ion-exchange resins, distillation, or salting out, or a combination thereof. Then, lactic acid may be isolated or purified from the recovered lactate. Highly pure L-lactic acid can be produced at a high yield by these procedures.

The fungus body of the genus Rhizopus subjected to the lactic acid production can be repetitively used for lactic acid production. Specifically, the fungus body can be recovered from the third culture medium after the recovery of lactic acid and then cultured again in the third culture medium in the same way as above to produce lactic acid again.

The method for producing lactic acid according to the present invention may be a batch method which involves alternately performing the culture of the fungus and the recovery of lactic acid accumulated in the culture medium followed by the replacement of the culture medium or may be a semibatch or continuous method which involves intermittently or continuously replacing a portion of the culture medium with a fresh one while concurrently performing the culture of the fungus and the recovery of lactic acid from the culture medium.

The present invention also encompasses the following composition, production method, use, or method as exemplary embodiments. However, the present invention is not intended to be limited by these embodiments.

<1> A method for improving the lactate dehydrogenase activity of a fungus of the genus Rhizopus, comprising the step of germinating a spore of a fungus of the genus Rhizopus in a first culture medium comprising 0.01% (w/v) or higher of a surfactant to obtain a mycelium.

<2> A method for producing a fungus of the genus Rhizopus having improved lactate dehydrogenase activity, comprising the step of

germinating a spore of a fungus of the genus Rhizopus in a first culture medium comprising 0.01% (w/v) or higher of a surfactant to obtain a mycelium.

<3> A method for improving the ability of a fungus of the genus Rhizopus to produce lactic acid, comprising the step of

germinating a spore of a fungus of the genus Rhizopus in a first culture medium comprising 0.01% (w/v) or higher of a surfactant to obtain a mycelium.

<4> A method for producing a fungus of the genus Rhizopus having improved ability to produce lactic acid, comprising the step of

germinating a spore of a fungus of the genus Rhizopus in a first culture medium comprising 0.01% (w/v) or higher of a surfactant to obtain a mycelium.

<5> The method according to any of <1> to <4>, preferably further comprising the step of growing the obtained mycelium in a second culture medium.

<6> A fungus of the genus Rhizopus produced by germinating a spore of a fungus of the genus Rhizopus in a first culture medium comprising 0.01% (w/v) or higher of a surfactant to obtain a mycelium.

<7> A fungus of the genus Rhizopus produced by: germinating a spore of a fungus of the genus Rhizopus in a first culture medium comprising 0.01% (w/v) or higher of a surfactant; and growing the obtained mycelium in a second culture medium.

<8> A method for producing lactic acid, comprising the steps of:
(A1) germinating a spore of a fungus of the genus Rhizopus in a first culture medium comprising 0.01% (w/v) or higher of a surfactant to obtain a mycelium having improved lactate dehydrogenase activity; and
(B) culturing the obtained mycelium in a third culture medium to obtain lactic acid.

<9> A method for producing lactic acid, comprising the steps of:
(A1) germinating a spore of a fungus of the genus Rhizopus in a first culture medium comprising 0.01% (w/v) or higher of a surfactant to obtain a mycelium having improved lactate dehydrogenase activity;
(A2) growing the mycelium obtained in the step (A1) in a second culture medium; and
(B) culturing the mycelium obtained in the step (A2) in a third culture medium to obtain lactic acid.

<10> The method according to <8> or <9>, preferably further comprising the step of recovering the produced lactic acid.

<11> The method or the fungus of the genus Rhizopus according to any of <1> to <10>, wherein the surfactant is

preferably at least one surfactant selected from the group consisting of sorbitan fatty acid ester, POE sorbitan fatty acid ester, POE alkyl ether having an EO addition mole number of 5 or less, deoxycholate, alkenyl succinate, and polyoxyethylene alkyl phenyl ether,

more preferably at least one surfactant selected from the group consisting of sorbitan monolaurate, sorbitan monooleate, polyoxyethylene sorbitan monolaurate having an EO average addition mole number of 20 or less, polyoxyethylene lauryl ether having an EO average addition mole number of 5 or less, deoxycholate, alkenyl succinate, and polyoxyethylene octyl phenyl ether having an EO average addition mole number of 10,

further preferably at least one surfactant selected from the group consisting of sorbitan monolaurate, sorbitan monooleate, polyoxyethylene sorbitan monolaurate having an EO average addition mole number of 4, polyoxyethylene lauryl ether having an EO average addition mole number of 3 or 4, and alkenyl succinate,

still further preferably sorbitan monolaurate.

<12> The method or the fungus of the genus Rhizopus according to any of <1> to <11>, wherein the surfactant in the first culture medium has a concentration of

preferably 0.01% (w/v) or higher, more preferably 0.05% (w/v) or higher, further preferably 0.1% (w/v) or higher, still further preferably 0.2% (w/v) or higher, and preferably lower than 2.5% (w/v), more preferably lower than 1.5% (w/v), further preferably lower than 1.0% (w/v), still further preferably lower than 0.8% (w/v), in terms of the final concentration in the culture medium, or

preferably 0.01% (w/v) or higher and lower than 2.5% (w/v), more preferably 0.05% (w/v) or higher and lower than 1.5% (w/v), further preferably 0.1% (w/v) or higher and lower than 1.0% (w/v), still further preferably 0.2% (w/v) or higher and lower than 0.8% (w/v), in terms of the final concentration in the culture medium.

<13> The method or the fungus of the genus Rhizopus according to any of <1> to <12>, wherein the fungus of the genus Rhizopus is preferably Rhizopus oryzae, more preferably selected from the group consisting of Rhizopus oryzae NBRC 4707, Rhizopus oryzae NBRC 4785, Rhizopus oryzae NBRC 5384, and Rhizopus oryzae NBRC 5418.

<14> The method or the fungus of the genus Rhizopus according to any of <5>, <7>, and <9> to <13>, wherein the surfactant in the second culture medium has a concentration of preferably 0.2% (w/v) or lower, more preferably 0.15% (w/v) or lower, further preferably 0.1% (w/v) or lower, still further preferably 0.05% (w/v) or lower, particularly preferably 0.01% (w/v) or lower.

<15> The method or the fungus of the genus Rhizopus according to any of <8> to <14>, wherein the surfactant in the third culture medium has a concentration of preferably 0.2% (w/v) or lower, more preferably 0.15% (w/v) or lower, further preferably 0.1% (w/v) or lower, still further preferably 0.05% (w/v) or lower, particularly preferably 0.01% (w/v) or lower.

<16> The method or the fungus of the genus Rhizopus according to any of <1> to <15>, wherein the spore of a fungus of the genus Rhizopus is cultured under the following conditions in the first culture medium:

stirring: preferably from 80 to 250 rpm, more preferably from 100 to 170 rpm

temperature: preferably from 25 to 42.5° C.

time: preferably from 24 to 120 hours, more preferably from 48 to 72 hours.

<17> The method or the fungus of the genus Rhizopus according to any of <5>, <7>, and <9> to <16>, wherein the mycelium is cultured under the following conditions in the second culture medium:

stirring: preferably from 100 to 300 rpm, more preferably from 170 to 230 rpm

temperature: preferably from 25 to 42.5° C.

time: preferably from 12 to 120 hours, more preferably from 24 to 72 hours.

<18> The method or the fungus of the genus Rhizopus according to any of <8> to <17>, wherein the mycelium is cultured under the following conditions in the third culture medium:

stirring: preferably from 100 to 300 rpm, more preferably from 170 to 230 rpm

temperature: preferably from 25 to 45° C.

time: preferably from 4 hours to 24 hours, more preferably from 6 hours to 12 hours.

### EXAMPLES

Hereinafter, the present invention will be described more specifically with reference to Examples.

### Production Example 1

### Method for Preparing Spore Suspension

A loopful of a cryopreserved spore suspension sample (−80° C.) of a Rhizopus oryzae JCM14625 strain (=NBRC 5384) obtained from a culture collection Riken BioResource Center (BRC) was inoculated to a PDA medium (Difco Potato Dextrose Agar, manufactured by Becton, Dickinson and Company) and then statically cultured at 30° C. for from 7 to 10 days. After visual confirmation of hyphal growth and blackening at the ends of hyphae associated with sporulation, from 30 to 40 mL of saline was added thereto. Spores, together with hyphae, were collected into a 50-mL centrifugal tube with a lid (manufactured by Greiner bio-one) using a platinum loop and then vigorously mixed in the tube. The spore suspension thus mixed was filtered through 3GP100 cylindrical funnel-shaped glass filter (manufactured by Shibata Scientific Technology Ltd.). The resulting filtrate was used as a spore solution. The number of spores in the spore solution was measured using a hemocytometer (D=1/50 mm· 1/400 mm²) after appropriate dilution with saline.

### Production Example 2

### Preparation of Mycelium

80 mL of a PDB medium non-supplemented or supplemented with 0.5% (w/v) (final concentration) of each surfactant described below was applied to a 200-mL baffled Erlenmeyer flask (manufactured by Asahi Glass Co., Ltd.). The Rhizopus oryzae spore suspension prepared in Production Example 1 was inoculated thereto at a dose of 1×10³ spores/mL of the medium and then cultured at 27° C. for 3 days with stirring at 170 rpm. Since mycelia would remain on the filter, the cultures were filtered through a stainless sieve of 250 μm in mesh size (manufactured by AS ONE Corp.) sterilized in advance to recover fungus bodies onto the filter.

Surfactant
Sorbitan monolaurate: Rheodol(R) SP-L10 (Kao Corp.)
Sorbitan monooleate: Rheodol(R) SP-010V (Kao Corp.)
Polyoxyethylene (3) lauryl ether: Emulgen(R) 103 (Kao Corp.)
Polyoxyethylene (4) lauryl ether: Emulgen(R) 104P (Kao Corp.)
Polyoxyethylene (5) lauryl ether: Emulgen(R) 106 (Kao Corp.)
Polyoxyethylene (6) lauryl ether: Emulgen(R) 108 (Kao Corp.)

Polyoxyethylene (4) sorbitan monolaurate: Rheodol(R) TW-L106 (Kao Corp.)
Polyoxyethylene (20) sorbitan monolaurate: Rheodol(R) TW-L120 (Kao Corp.)
Sodium deoxycholate (Wako Pure Chemical Industries Ltd.)
Potassium alkenyl succinate (28%): Latemul(R) ASK (Kao Corp.)
Polyoxyethylene (10) octyl phenyl ether: TRITON(R) X-100 (MP Biomedicals, LLC)
   (The number within the parentheses following the term “polyoxyethylene” in the name of each surfactant represents an EO average addition mole number.)

### Production Example 3

### Growth of Mycelium

From3.0 to 4.0 g (wet weight) of the recovered fungus bodies was inoculated to 100 mL of an inorganic culture medium (composition: 10% glucose, 0.1% ammonium sulfate, 0.06% potassium dihydrogen phosphate, 0.025% magnesium sulfate heptahydrate, 0.009% zinc sulfate heptahydrate, and 5.0% calcium carbonate; all the concentrations mean % (w/v)) applied to a 500-mL Erlenmeyer flask, and then cultured at 27° C. for approximately 40 hours with stirring at 220 rpm. Subsequently, the cultures thus obtained by culture in the inorganic culture medium were filtered using a stainless screen filter holder (manufactured by EMD Millipore) sterilized in advance to recover fungus bodies onto the filter. On this filter holder, the fungus bodies were further washed with 100 mL of saline. The saline used in washing was removed by suction filtration. The obtained fungus bodies were used in the following LDH activity evaluation and evaluation of the ability to fermentatively produce lactic acid.

### Test Example 1

### LDH Activity Evaluation—1

6.0 g of the wet fungus bodies of the genus Rhizopus obtained in Production Example 3 was inoculated to 40 mL of an inorganic culture medium for lactic acid production evaluation (composition: 8.0% glucose, 0.1% ammonium sulfate, 0.06% potassium dihydrogen phosphate, 0.025% magnesium sulfate heptahydrate, 0.009% zinc sulfate heptahydrate, and 5.0% calcium carbonate; all the concentrations mean % (w/v)) applied to a 200-mL Erlenmeyer flask, and then cultured at 35° C. with stirring at 170 rpm. After 4.5-hour culture of the fungus bodies of the genus Rhizopus with stirring, the fungus bodies were filtered using a stainless screen filter holder (manufactured by EMD Millipore) sterilized in advance to recover fungus bodies onto the filter. On this filter holder, the fungus bodies were further washed with 100 mL of saline. The saline used in washing was removed by suction filtration. 0.3 g of the obtained fungus bodies was recovered into a 3-mL disruption tube (manufactured by Yasui Kikai Corp.). Metal cone for 3 mL (manufactured by Yasui Kikai Corp.) was added to the tube, which was then capped and then frozen in liquid nitrogen. The frozen 3-mL disruption tube was applied to Multi-Beads Shocker (manufactured by Yasui Kikai Corp.). The fungus bodies were disrupted at 1700 rpm for 10 seconds. Then, 1 mL of 0.1 M Tris-HCl (pH 7.5) was added to the tube, followed by treatment in Multi-Beads Shocker at 1700 rpm for 10 seconds. The treated solution was centrifuged at 15000 rpm at 4° C. for 5 minutes. The obtained supernatant was used as a fungus body extract.

The LDH activity refers to the activity of converting pyruvate to lactate. This enzymatic reaction requires NADH as a coenzyme. 10 μL of the fungus body extract appropriately diluted was mixed with 150 μL of a reaction solution (composition: 0.1 M Tris-HCl and 700 μM NADH), and the mixture was then left standing at 30° C. for 5 minutes. The enzymatic reaction was initiated by the addition thereto of 40 μL of a 20 mM sodium pyruvate solution preincubated to 30° C. The absorbance of this reaction solution was measured at 340 nm to assay the LDH activity (see Non Patent Literature 4). 1 U was defined as the amount of an enzyme which produces 1 μmol NAD⁺ for 1 minute. Protein concentrations were measured using BSA as a standard and Protein Assay Dye Reagent Concentrate (Bio-Rad Laboratories, Inc.).

The results are shown in Table 1. The results are indicated by a relative value with LDH activity in the absence of the surfactant defined as 100. The fungus bodies of the genus Rhizopus having mycelia prepared from spores in the medium supplemented with sorbitan monolaurate, sorbitan monooleate, polyoxyethylene sorbitan monolaurate having an EO average addition mole number of 20 or less, polyoxyethylene lauryl ether having an EO average addition mole number of 5 or less, sodium deoxycholate, alkenyl succinate, or polyoxyethylene octyl phenyl ether having an EO average addition mole number of 10 had higher LDH activity than that of the control fungus bodies of the genus Rhizopus having mycelia prepared from spores in the medium non-supplemented with the surfactant. Also, the fungus bodies of the genus Rhizopus having mycelia prepared from spores in the medium supplemented with polyoxyethylene lauryl ether having an EO average addition mole number of 6 were confirmed to have slightly lower LDH activity than that of the control fungus bodies of the genus Rhizopus havingmycelia prepared from spores in the medium non-supplemented with the surfactant.

TABLE 1

| | LDH activity |
|---|---|
| Surfactant | (relative value) |
| Sorbitan monolaurate | 267 |
| Sorbitan monooleate | 135 |
| Polyoxyethylene (3) lauryl ether | 170 |
| Polyoxyethylene (4) lauryl ether | 214 |
| Polyoxyethylene (5) lauryl ether | 112 |
| Polyoxyethylene (4) sorbitan monolaurate | 140 |
| Polyoxyethylene (20) sorbitan monolaurate | 148 |
| Sodium deoxycholate | 109 |
| Potassium alkenyl succinate (28%) | 381 |
| Polyoxyethylene (10) octyl phenyl ether | 178 |
| No surfactant added | 100 |
| Polyoxyethylene (6) lauryl ether | 93 |

### Test Example 2

### Evaluation of Ability to Produce Lactic Acid—1

Each surfactant which improved the LDH activity by 1.3 times or more compared with the control in Test Example 1 was used to prepare mycelia of a fungus of the genus Rhizopus and evaluate the ability to produce lactic acid.

Specifically, the mycelia of a fungus of the genus Rhizopus were prepared by the method of Production Example 2 using the surfactant and then grown by the method of Production Example 3. 6.0 g of the obtained wet fungus bodies of the genus Rhizopus was inoculated to 40 mL of an inorganic culture medium for lactic acid production evaluation (composition: 8.0% glucose, 0.1% ammonium sulfate, 0.06% potassium dihydrogen phosphate, 0.025% magnesium sulfate heptahydrate, 0.009% zinc sulfate heptahydrate, and 5.0% calcium carbonate; all the concentrations mean % (w/v)) applied to a 200-mL Erlenmeyer flask, and then cultured at 35° C. with stirring at 170 rpm. The supernatant of the culture medium was recovered every 2 hours from 30 minutes after the fungus body inoculation. Glucose and lactic acid were quantified by the procedures described later in Reference Example 1. A relative value of the production rate of lactic acid was determined on the basis of the calculation method described in Reference Example 2.

The results are shown in Table 2. The results are indicated by a relative value with the production rate of lactic acid in the absence of the surfactant defined as 100. All the fungus bodies of the genus Rhizopus having mycelia prepared from spores in the medium supplemented with each surfactant which exhibited high LDH-improving activity in Test Example 1 had the higher ability to produce lactic acid than that of the control fungus bodies of the genus Rhizopus having mycelia prepared from spores in the medium non-supplemented with the surfactant.

TABLE 2

| | Production rate of |
|---|---|
| | lactic acid |
| Surfactant | (relative value) |
| Sorbitan monolaurate | 152 |
| Sorbitan monooleate | 113 |
| Polyoxyethylene (3) lauryl ether | 203 |
| Polyoxyethylene (4) lauryl ether | 171 |
| Polyoxyethylene (4) sorbitan monolaurate | 142 |
| Polyoxyethylene (20) sorbitan monolaurate | 137 |
| Potassium alkenyl succinate (28%) | 177 |
| Polyoxyethylene (10) octyl phenyl ether | 254 |
| No surfactant added | 100 |

### Test Example 3

### Evaluation of Foaming Property

Each surfactant used in Test Example 2 was evaluated for its foaming properties by the inversion stirring method. 100 mL of a PDB medium non-supplemented or supplemented with 0.5% (w/v) (final concentration) of a surfactant was added to a graduated cylindrical container of 50 mm in diameter and then inversion-stirred for 300 seconds using a flat propeller while the propeller was inverted every 6 seconds at 1000 rpm. 30 seconds after the completion of stirring, the volume of foam in the medium was measured.

The results are shown in Table 3. The results are indicated by a relative value with the volume of foam in the absence of the surfactant defined as 100. Sorbitan monolaurate (Rheodol(R) SP-L10), sorbitan monooleate (Rheodol(R) SP-010V), polyoxyethylene (3) lauryl ether (Emulgen(R) 103), polyoxyethylene (4) lauryl ether (Emulgen(R) 104P), polyoxyethylene (4) sorbitan monolaurate (Rheodol(R) TW-L106), and potassium alkenyl succinate (28%) (Latemul(R) ASK) produced 6 times or less the volume of foam in the absence of the surfactant and were thus considered suitable for culture. Particularly, sorbitan monolaurate (Rheodol(R) SP-L10), sorbitan monooleate (Rheodol(R) SP-010V), and polyoxyethylene (4) sorbitan monolaurate (Rheodol(R) TW-L106) produced twice or less the volume of foam in the absence of the surfactant and were thus considered more preferably suitable for culture.

TABLE 3

| | Volume of foam |
|---|---|
| Surfactant | (relative value) |
| Sorbitan monolaurate | 150 |
| Sorbitan monooleate | 100 |
| Polyoxyethylene (3) lauryl ether | 250 |
| Polyoxyethylene (4) lauryl ether | 350 |
| Polyoxyethylene (4) sorbitan monolaurate | 150 |
| Polyoxyethylene (20) sorbitan monolaurate | 1350 |
| Potassium alkenyl succinate (28%) | 600 |
| Polyoxyethylene (10) octyl phenyl ether | 2000 |
| No surfactant added | 100 |

### Test Example 4

### Evaluation of Ability to Produce Lactic Acid—2

### (Preparation of Mycelium)

(1) Spore suspensions were prepared in the same way as in Production Example 1 using Rhizopus oryzae NBRC 4707, NBRC 4785, NBRC 5384 (=JCM14625), and NBRC 5418 strains obtained from a culture collection NBRC.

(2) Mycelia were prepared from the spores of each strain under the same conditions as in Production Example 2 involving stirring at 100 rpm using 60 mL of a PDB medium non-supplemented or supplemented with 0.5% (w/v) (final concentration) of the surfactant sorbitan monolaurate (Rheodol(R) SP-L10, manufactured by Kao Corp.).

(3) The total amount of the fungus bodies obtained in (2) was inoculated to 100 mL of an inorganic culture medium under the same conditions as in Production Example 3 to grow the mycelia. Control conditions in the absence of the surfactant involved culturing spores with stirring by the same procedures as above using a PDB medium non-supplemented with the surfactant and then inoculating from 4 to 8 mL of the PDB seed culture medium to 100 mL of the same inorganic culture medium as above.

### (Evaluation of Ability to Produce Lactic Acid)

30 g of the prepared wet fungus bodies of each strain of the genus Rhizopus was inoculated to 100 mL of an inorganic culture medium for lactic acid production evaluation (composition: 12.5% glucose, 0.1% ammonium sulfate, 0.06% potassium dihydrogen phosphate, 0.025% magnesium sulfate heptahydrate, 0.009% zinc sulfate heptahydrate, and 6.25% calcium carbonate; all the concentrations mean % (w/v)) applied to a 500-mL Erlenmeyer flask, and then cultured at 33.5° C. with stirring at 170 rpm. The production rate of lactic acid was determined by the same procedures as in Test Example 2.

The results are shown in Table 4. The results are indicated by a relative value with the production rate of lactic acid by each fungus strain under control conditions (in the absence of the surfactant) defined as 100. All the mycelia prepared from the spores of the fungus strains in the surfactant-supplemented medium had the improved ability to produce lactic acid.

TABLE 4

| | Production rate of |
|---|---|
| | lactic acid |
| Fungus strain | (relative value) |
| NBRC 4707 | 135 |
| NBRC 4785 | 170 |
| NBRC 5384 | 130 |
| NBRC 5418 | 135 |

### Test Example 5

### Evaluation of Ability to Produce Lactic Acid—3

### (Preparation of Mycelium)

(1) 200 mL of a PDB medium non-supplemented or supplemented with 0.01, 0.05, 0.25, 0.50, 1.0, 1.5, or 2.5% (w/v) (final concentration) of sorbitan monolaurate (Rheodol(R) SP-L10, manufactured by Kao Corp.) was applied to a 500-mL baffled Erlenmeyer flask (manufactured by Asahi Glass Co., Ltd.). The Rhizopus oryzae spore suspension prepared in Production Example 1 was inoculated thereto at a dose of 1×10³ spores/mL of the medium and then cultured at 27° C. for 3 days with stirring at 110 rpm.

(2) The cultures were filtered through a filter support and a nylon net filter of 180 μm mesh size (manufactured by EMD Millipore) sterilized in advance to recover fungus bodies onto the filter. From 2.0 to 3.0 g of the recovered fungus bodies was inoculated to 100 mL of an inorganic culture medium (composition: 10% glucose, 0.1% ammonium sulfate, 0.06% potassium dihydrogen phosphate, 0.025% magnesium sulfate heptahydrate, 0.009% zinc sulfate heptahydrate, and 5.0% calcium carbonate; all the concentrations mean % (w/v)) applied to a 500-mL Erlenmeyer flask, and then cultured at 27° C. for approximately 40 hours with stirring at 220 rpm.

(3) Control conditions in the absence of the surfactant involved culturing spores with stirring by the same procedures as in (1) using a PDB medium non-supplemented with the surfactant and then inoculating from 4 to 8 mL of the PDB seed culture medium to 100 mL of the inorganic culture medium by the same procedures as in (2), followed by culture.

(4) Subsequently, the cultures thus obtained by culture in the inorganic culture medium were filtered through a filter support and a nylon net filter of 180 μm mesh size (manufactured by EMD Millipore) sterilized in advance to recover fungus bodies onto the filter. On this filter, the fungus bodies were further washed with from 50 to 200 mL of saline to obtain fungus bodies.

### (Evaluation of Ability to Produce Lactic Acid)

30 g of the prepared wet fungus bodies of the genus Rhizopus was inoculated to 100 mL of an inorganic culture medium for lactic acid production evaluation (composition: 12.5% glucose, 0.1% ammonium sulfate, 0.06% potassium dihydrogen phosphate, 0.025% magnesium sulfate heptahydrate, 0.009% zinc sulfate heptahydrate, and 6.25% calcium carbonate; all the concentrations mean % (w/v)) and then cultured at 33.5° C. with stirring at 170 rpm. The fungus body-free supernatant of the culture medium was recovered every 1 hour from 30 minutes after the fungus body inoculation. Glucose and lactic acid were quantified by the procedures described later in Reference Example 1. A relative value of the production rate of lactic acid was determined on the basis of the calculation method described in Reference Example 2.

The results are shown in Table 5. The results about the ability to produce lactic acid shown in Table 5 are indicated by a relative value with results in the absence of the surfactant (control) defined as 100. The mycelia prepared from spores in the medium supplemented with the surfactant in a concentration range of 0.01% (w/v) or higher and lower than 2.5% (w/v) had the higher or equivalent ability to produce lactic acid compared with the control.

TABLE 5

| Sorbitan monolaurate | Production rate of |
|---|---|
| concentration | lactic acid |
| (% w/v) | (relative value) |
| No surfactant added | 100 |
| 0.01 | 119 |
| 0.05 | 133 |
| 0.25 | 116 |
| 0.50 | 138 |
| 1.00 | 140 |
| 1.50 | 117 |
| 2.50 | 98 |

### Test Example 6

### Evaluation of Ability to Produce Lactic Acid by Repetitive Use of Fungus Body

Mycelia of a fungus of the genus Rhizopus were prepared in a medium non-supplemented with or containing 0.5% (w/v) (final concentration) of the surfactant sorbitan monolaurate (Rheodol(R) SP-L10) by the same procedures as in Test Example 4(2) and then cultured by the same procedures as in Test Example 4(3).

The obtained fungus bodies were recovered by the same procedures as in Test Example 5(4). Subsequently, 25 g of the wet fungus bodies was cultured for 4 hours in an inorganic culture medium for lactic acid production evaluation by the same procedures as in Test Example 5. The production rate of lactic acid was determined. The same procedures of fungus body recovery and culture were repeated, and the production rate of lactic acid was determined at each run (the number of repetitions: 3). The production rate of lactic acid at each run was calculated as a relative value to the production rate of lactic acid under the control conditions (in the absence of the surfactant) in the first repetition.

The results are shown in Table 6. The fungus body pellet of the genus Rhizopus for lactic acid production of the present invention maintained its high ability to produce lactic acid even by repetitive use.

TABLE 6

| | Production rate of |
|---|---|
| The number of | lactic acid |
| repetition | (relative value) |
| 1 | 173 |
| 2 | 170 |
| 3 | 167 |

### Test Example 7

### Evaluation of Ability to Produce Lactic Acid—4

Mycelia of a fungus of the genus Rhizopus were prepared in a medium containing 0.5% (w/v) (final concentration) of sorbitan monolaurate (Rheodol(R) SP-L10) by the same procedures as in Production Example 2 and then cultured by the same procedures as in Production Example 3. The fungus bodies were cultured by the sane procedures as in Test Example 2 and evaluated for its production rate of lactic acid. In this respect, the inorganic medium for lactic acid production evaluation was non-supplemented or supplemented with 0.1% (w/v), 0.2% (w/v), 0.5% (w/v), or 1% (w/v) (final concentration) of Rheodol(R) SP-L10. The amount of lactic acid was measured at 4 hours and 8.5 hours of the culture to calculate the production rate of lactic acid. The results are shown in Table 7. The production rate of lactic acid was reduced in the medium supplemented with 0.1% (w/v) or higher of Rheodol(R) SP-L10 during lactic acid production.

TABLE 7

| | Production rate of |
|---|---|
| Rheodol SP-L10 | lactic acid |
| concentration (% w/v) | (relative value) |
| 0 | 100 |
| 0.1 | 93 |
| 0.2 | 67 |
| 0.5 | 4 |
| 1 | 0 |

### Reference Example 1

### Quantification of Glucose and Lactic Acid in Culture for Evaluation of Ability to Produce Lactic Acid

Glucose and lactic acid were quantified using an HPLC apparatus LaChrom Elite (manufactured by Hitachi High-Technologies Corp.). The analytical column used was a carbohydrate/organic acid analysis column HPX-87H (7.8 mm I.D.×30 cm, manufactured by Bio-Rad Laboratories, Inc.) connected with a guard column Cation H (4.6 mm I.D.×3.0 cm, manufactured by Bio-Rad Laboratories, Inc.). Elution was performed under conditions involving 10 mM sulfuric acid as an eluent, a flow rate of 0.85 mL/min, and a column temperature of 50° C. Glucose and lactic acid were detected using a differential refractive index detector (RI detector) and a UV detector (detection wavelength: 210 nm), respectively. The standard samples used were glucose (distributor code: 049-31165, manufactured by Wako Pure Chemical Industries Ltd.), and lithium L-lactate (product number: L2250, manufactured by Sigma-Aldrich Inc.). Quantification was performed on the basis of concentration calibration curves prepared using these standard samples.

The supernatant sample of each culture medium to be subjected to HPLC analysis was appropriately diluted in advance with 37 mM sulfuric acid and then applied to DISMIC-13cp (0.20 μm cellulose acetate membrane, manufactured by ADVANTEC Group) or MULTI SCREEN MNHV45 (0.45 μm Durapore membrane, manufactured by EMD Millipore) to remove insoluble matter.

### Reference Example 2

### Method for Calculating Production Rate of Lactic Acid

The concentration of lactic acid (unit: g of lactic acid/L of medium) in the supernatant sample of each culture medium was measured according to Reference Example 1 and divided by a time (unit: hr) required for change in lactic acid concentration to calculate the production rate of lactic acid per unit time (unit: g of lactic acid/L/hr). In addition, a relative value to the production rate of lactic acid under control conditions in each Production Example was determined.

What is claimed is:

1. A method for producing lactic acid, comprising the following steps (A1) and (B):
(A1) germinating a spore of a fungus of the genus Rhizopus in a first culture medium comprising 0.01% (w/v) or higher of a surfactant to obtain a mycelium having improved lactate dehydrogenase activity; and
(B) further culturing the obtained mycelium in a third culture medium to obtain lactic acid, wherein
the surfactant is at least one surfactant selected from the group consisting of sorbitan fatty acid ester, POE sorbitan fatty acid ester, POE alkyl ether having an EO addition mole number of 5 or less, deoxycholate, alkenyl succinate, and polyoxyethylene alkyl phenyl ether.

2. The method according to claim 1, wherein the surfactant in the third culture medium has a concentration of 0.2% (w/v) or lower.

3. The method according to claim 1, wherein the surfactant in the first culture medium has a concentration of 0.01% (w/v) or higher and lower than 2.5% (w/v).

4. The method according to claim 1, wherein the surfactant is at least one surfactant selected from the group consisting of sorbitan monolaurate, sorbitan monooleate, polyoxyethylene sorbitan monolaurate having an EO average addition mole number of 20 or less, polyoxyethylene lauryl ether having an EO average addition mole number of 5 or less, deoxycholate, alkenyl succinate, and polyoxyethylene octyl phenyl ether having an EO average addition mole number of 10.

5. The method according to claim 1, further comprising the following step (A2) between step (A1) and step (B):
(A2) growing the mycelium obtained in step (A1) in a second culture medium.

6. The method according to claim 5, wherein the surfactant in the second culture medium has a concentration of 0.2% (w/v) or lower.

7. The method according to claim 1, wherein the fungus of the genus Rhizopus is a Rhizopus oryzae strain.

8. The method according to claim 7, wherein the Rhizopus oryzae strain is selected from the group consisting of Rhizopus oryzae NBRC 4707, Rhizopus oryzae NBRC 4785, Rhizopus oryzae NBRC 5384, and Rhizopus oryzae NBRC 5418.

9. A method for improving the lactate dehydrogenase activity of a fungus of the genus Rhizopus, comprising the following step (A1):
(A1) germinating a spore of a fungus of the genus Rhizopus in a first culture medium comprising 0.01% (w/v) or higher of a surfactant to obtain a mycelium, wherein the surfactant is at least one surfactant selected from the group consisting of sorbitan fatty acid ester, POE sorbitan fatty acid ester, POE alkyl ether having an EO addition mole number of 5 or less, deoxycholate, alkenyl succinate, and polyoxyethylene alkyl phenyl ether.

10. The method according to claim 9, wherein the surfactant in the first culture medium has a concentration of 0.01% (w/v) or higher and lower than 2.5% (w/v).

11. The method for improving the activity of lactate dehydrogenase activity of fungus of the genus Rhizopus according to claim 9, wherein the surfactant is at least one surfactant selected from the group consisting of sorbitan monolaurate, sorbitan monooleate, polyoxyethylene sorbitan monolaurate having an EO average addition mole number of 20 or less, polyoxyethylene lauryl ether having an EO average addition mole number of 5 or less, deoxycholate, alkenyl succinate, and polyoxyethylene octyl phenyl ether having an EO average addition mole number of 10.

12. The method for improving the activity of lactate dehydrogenase activity of fungus of the genus Rhizopus according to claim 9, further comprising the following step (A2) after step (A1):
(A2) growing the mycelium obtained in step (A1) in a second culture medium.

13. The method for improving the activity of lactate dehydrogenase activity of fungus of the genus Rhizopus according to claim 12, wherein the surfactant in the second culture medium has a concentration of 0.2% (w/v) or lower.

14. The method for improving the activity of lactate dehydrogenase activity of fungus of the genus Rhizopus according to claim 9, wherein the fungus of the genus Rhizopus is a Rhizopus oryzae strain.

15. The method for improving the activity of lactate dehydrogenase activity of fungus of the genus Rhizopus according to claim 14, wherein the Rhizopus oryzae strain is selected from the group consisting of Rhizopus oryzae NBRC 4707, Rhizopus oryzae NBRC 4785, Rhizopus oryzae NBRC 5384, and Rhizopus oryzae NBRC 5418.

16. A method for producing a fungus of the genus Rhizopus having improved ability to produce lactic acid, comprising obtaining a fungus of the genus Rhizopus having improved lactate dehydrogenase activity by a method according to claim 9.

17. (canceled)

18. The method according to claim 1, wherein the mycelia of the fungus of the genus Rhizopus obtained by the above step (A1) have a pellet form.

19. The method according to claim 5, wherein the mycelia of the fungus of the genus Rhizopus obtained by the above step (A2) have a pellet form.

20. The method according to claim 9, wherein the mycelia of the fungus of the genus Rhizopus obtained by the above step (A1) have a pellet form.

21. The method according to claim 12, wherein the mycelia of the fungus of the genus Rhizopus obtained by the above step (A2) have a pellet form.
